(19) United States
(12) Patent Application Publication
(10) Pub. No.: US 2013/0253205 A1
(43) Pub. Date: Sep. 26, 2013

(54) PROCESS FOR PREPARATION OF AMINOCYCLOHEXYL ETHERS AND INTERMEDIATE PRODUCTS USED IN THE PROCESS

(71) Applicants: Boris Gorin, Oakville (CA); Craig Edward Dixon, Brooklin (CA); Jan Oudenes, Aurora (CA); Elena Valentina Bejan, Brantford (CA); Aaron Cleveland Kinsman, Mississauga (CA)

(72) Inventors: Boris Gorin, Oakville (CA); Craig Edward Dixon, Brooklin (CA); Jan Oudenes, Aurora (CA); Elena Valentina Bejan, Brantford (CA); Aaron Cleveland Kinsman, Mississauga (CA)

(73) Assignee: ALPHORA RESEARCH INC., Mississauga, ON (CA)

(21) Appl. No.: 13/825,979

(22) PCT Filed: Sep. 26, 2011
(86) PCT No.: PCT/CA2011/050598

§ 371 Date: Jun. 12, 2013

Related U.S. Application Data
(60) Provisional application No. 61/386,830, filed on Sep. 27, 2010.

(51) Int. Cl. C07D 207/12; C07F 7/18

(52) U.S. Cl. CPC C07D 207/12; C07F 7/1856; C07F 7/188
USPC 548/406; 548/541

(57) ABSTRACT

A process for preparation of a compound of formula (I) or or a pharmaceutically acceptable salt, ester, or prodrug thereof, is disclosed. The process involves hydrogenating, in the presence of a catalyst, a compound of formula (II). The different substituents are as described in the specification. Also disclosed are intermediates and processes for their preparation. Further, the process can provide an alternate route for the synthesis of Vernakalant from starting materials that can be readily available.

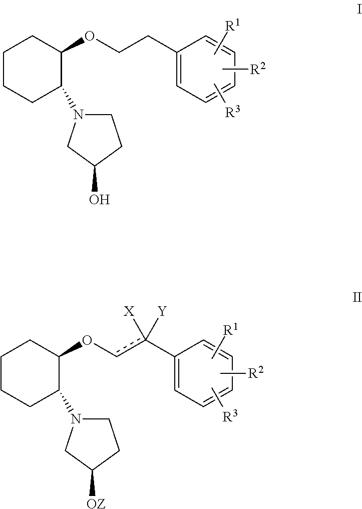

### TECHNICAL FIELD

The specification relates to a process for preparation of aminocyclohexyl ethers and intermediate products used in the process.

### BACKGROUND

WO 99/50225 and WO 2004/099137 disclose aminocyclohexyl ether compounds as being useful in the treatment of arrhythmias. Some of the compounds disclosed therein have been found to be effective in the treatment and/or prevention of atrial fibrillation (AF). The process for the preparation of the compounds disclosed can involve a complex synthetic route, including multiple protection and deprotection steps.

Among the aminocyclohexyl ether compounds, the compound (1R, 2R)-2-[(3R)-Hydroxypyrrolidinyl]-1-(3,4-dimethyoxyphenethoxy)-cyclohexane, which has also been named as (3R)-1-{(1R,2R)-2-[2-(3,4-dimethoxyphenyl)ethoxy]cyclohexyl}pyrrolidin-3-ol, and known as Vernakalant, shown below, has been taught to be useful for the treatment of atrial fibrillation.

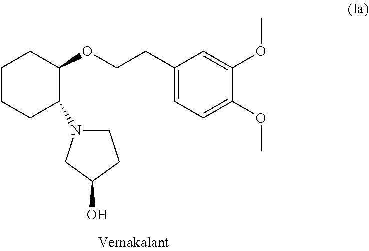 [SMILES: COc1ccc(CCO[C@@H]2CCCC[C@H]2N2CC[C@@H](O)C2)cc1OC]

WO 2006/138673 discloses a process for the preparation of compounds of formula A or B, as shown below, and their individual stereoisomers. The process disclosed can require separation of stereoisomers near the end of the synthetic route, which can be challenging and at times practically unfeasible. In addition, the synthetic route disclosed involves multiple synthetic steps, including formation of a tertiary cyclic amine moiety of the compounds of formula A or B, which may not be commercially viable as an industrial process.

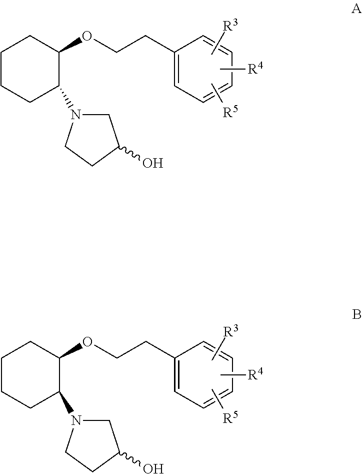

In addition, the process disclosed in the above patent applications can require multiple protection and deprotection steps in the synthetic process. Such protection and deprotection steps can increase the amount of product handling and can affect the product yield and cost. It would be desirable to have a synthetic process having a reduced number of protection and deprotection steps. It would also be desirable to have a process, where a single global deprotection step is performed to obtain the desired compound.

Therefore, an alternate route for the synthesis of such aminocyclohexyl ether compounds, including Vernakalant, and their pharmaceutically acceptable salts, can be useful. In addition, a process where the compounds are prepared using readily available starting materials can be useful. Moreover, a process that allows a single global deprotection step as the last step or near the last steps of the synthetic process can be useful.

### SUMMARY OF THE INVENTION

In one aspect, the specification relates to a process for preparation of aminocyclohexyl ether of formula I

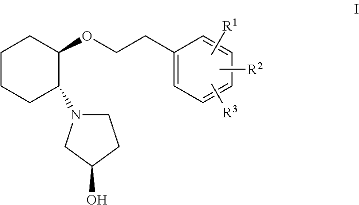

or a pharmaceutically acceptable salt, ester, or prodrug thereof. The process comprising hydrogenating, in the presence of a catalyst, a compound of formula II

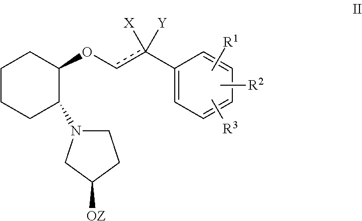

where R¹, R², R³, X, Y, Z and [symbol] are as defined herein.

In another aspect, the specification relates to a compound of formula II

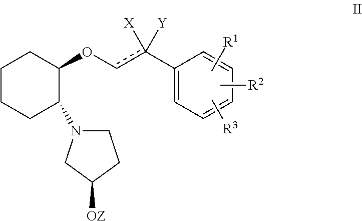

and process for its preparation.

### DETAILED DESCRIPTION

As noted above, in one aspect the specification relates to a process for preparation of aminocyclohexyl ether of formula I

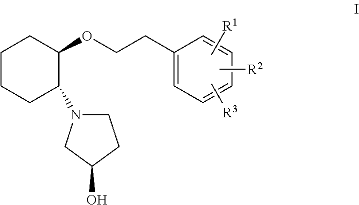

or a pharmaceutically acceptable salt, ester, or prodrug thereof; wherein:
R¹, R² and R³ each independently is bromine, chlorine, fluorine, carboxy, hydrogen, hydroxy, hydroxy methyl, methanesulfonamido, cyano, sulfamyl, trifluoromethyl, —CHF₂, —SO₂N(R⁶)R⁷, —OCF₃, C₁₋₆alkyl, C₁₋₆alkoxy, C₂₋₇alkoxycarbonyl, C₂-C₇alkanoyloxy, aryl or —N(R⁴)R⁵; with the proviso that at least one of R¹, R² or R³ is other than hydrogen; and
R⁴, R⁵, R⁶ and R⁷ each independently is hydrogen, acetyl, methanesulfonyl or C₁₋₆alkyl;
   the process comprising:

hydrogenating, in the presence of a catalyst, a compound of formula II

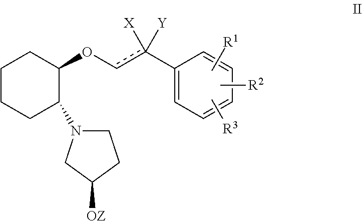

wherein X and Y each independently is hydrogen, hydroxyl, amino, C₁₋₆alkoxy, C₆₋₁₄aryloxy, C₁₋₆alkylamino, C₆₋₁₄arylamino, or silyloxy, or X and Y together with the carbon atom to which they are attached form C═O, with the proviso that at least one of X and Y is other than hydrogen;

Z is a hydroxyl protecting group; and

[symbol] is a single or double bond, and wherein when [symbol] is a double bond, one of X or Y is absent and the one of X or Y present is other than hydrogen; and

optionally converting the aminocyclohexyl ether of formula I into its pharmaceutically acceptable salt, ester, or prodrug thereof.

In one embodiment, the process provides preparation of a diastereoisomer of a compound of formula II, where the cyclohexyl ether oxygen and nitrogen attached to the vicinal carbon of the cyclohexyl ether are in the opposite trans configuration shown in the compound of formula II.

In another embodiment, the process further comprises reacting a compound of formula III

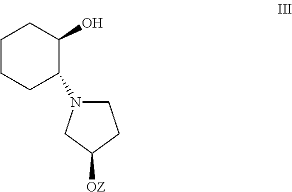

with an epoxide of formula IV

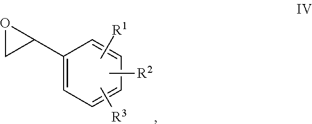

to form the compound of formula II, and

optionally silylating the reaction product to form the silyloxy derivative.

In another embodiment according to the specification, the compound of formula III is prepared by reacting a cyclohexyl epoxide of formula V

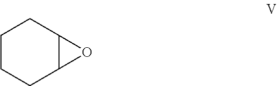

with a compound of formula VI, shown below, followed by chiral resolution.

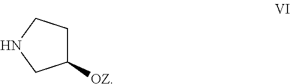

In another embodiment according to the specification, the compound of formula I prepared according to the process is a compound of formula Ia

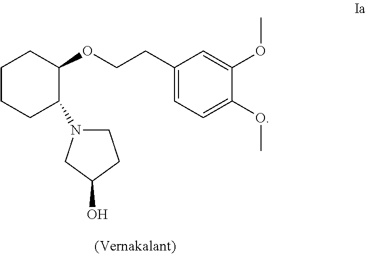 [SMILES: COc1ccc(CCO[C@@H]2CCCC[C@H]2N2CC[C@@H](O)C2)cc1OC]

Pharmaceutically acceptable salts in accordance with the specification are not particularly limited and should be known to a person of skill in the art or can be determined. The pharmaceutically acceptable salt according to the specification can be obtained from the combination of the compound of formula I and a pharmaceutically acceptable organic or inorganic acid (acid addition salts) which retain the biological effectiveness and properties of the compounds and which are not biologically or otherwise undesirable. Suitable acid addition salts can be obtained from the treatment with a mineral acid that include, for example and without limitation, hydrochloric acid, hydrobromic acid, phosphoric acid and sulfuric acid, or with an organic acid that include, for example and without limitation, ascorbic acid, citric acid, tartaric acid, lactic acid, maleic acid, malonic acid, fumaric acid, glycolic acid, succinic acid, propionic acid, acetic acid and methane sulfonic acid.

Pharmaceutically acceptable esters in accordance with the specification are not particularly limited and should be known to a person of skill in the art or can be determined. The pharmaceutically acceptable esters in accordance with the specification can be prepared by reacting, a hydroxy functional group with a pharmaceutically acceptable organic acid. The pharmaceutically acceptable organic acid can include, for example and without limitation, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid or salicylic acid.

A prodrug in accordance with the specification is not particularly limited and should be known to a person of skill in the art or can be determined. A prodrug is a drug which has been chemically modified and may be biologically inactive at its site of action, but which is degraded or modified by one or more enzymatic or other in vivo processes to the parent bioactive form. Generally, a prodrug can have a different pharmacokinetic profile than the parent drug such that, for example and without limitation, it is more easily absorbed across the mucosal epithelium, it has better salt formation or solubility and/or it has better systemic stability (e.g., an increased plasma half-life). Those skilled in the art recognize that chemical modifications of a parent drug to yield a prodrug include, for example and without limitation: (1) terminal ester or amide derivatives, which are susceptible to being cleaved by esterases or lipases; (2) terminal peptides, which may be recognized by specific or nonspecific proteases; or (3) a derivative that causes the prodrug to accumulate at a site of action through membrane selection, and combinations of the above techniques. Other non-limiting examples of prodrugs can include an acetate, pivaloate or benzoate of the parent drug.

The term C₁₋₆alkyl in accordance with the specification is not particularly limited and should be known to a person of skill in the art. The C₁₋₆ alkyl may be, for example, and without limitation, any straight or branched alkyl, for example, methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl or 3-methylpentyl.

The term C₁₋₆alkoxy in accordance with the specification is not particularly limited and should be known to a person of skill in the art. The C₁₋₆alkoxy is a C₁₋₆ alkyl group as described above, which is linked to an oxygen atom. For example, and without limitation, the C₁₋₆ alkoxy group may be methoxy, ethoxy, n-propoxy, i-propoxy and the like.

The term C₂₋₇alkanoyloxy in accordance with the specification is not particularly limited and should be known to a person of skill in the art. The term “alkanoyloxy” refers to an ester substituent wherein the non-carbonyl oxygen is the point of attachment to the molecule. Examples of alkanoyloxy can include, without limitation, propanoyloxy [(CH₃CH₂C(═O)—O—, a C₃-alkanoyloxy] and ethanoyloxy [CH₃C(═O)—O—, a C₂-alkanoyloxy].

The term C₂-C₇alkoxycarbonyl in accordance with the specification is not particularly limited and should be known to a person of skill in the art. The term “alkoxycarbonyl” refers to an ester substituent wherein the carbonyl carbon is the point of attachment to the molecule. Examples of alkoxycarbonyl can include, without limitation, ethoxycarbonyl [CH₃CH₂OC(═O)—, a C₃-alkoxycarbonyl] and methoxycarbonyl [CH₃C(═O)—, a C₂-alkoxycarbonyl].

The term aryl in accordance with the specification is not particularly limited and should be known to a person of skill in the art. The term “aryl” refers to aromatic groups which have at least one ring having a conjugated n-electron system and includes carbocyclic aryl, heterocyclic aryl (also known as heteroaryl groups) and biaryl groups, all of which may be optionally substituted. The aryl groups can include, for example and without limitation, six to fourteen atoms. Examples of aryl group can include, without limitation, phenyl, pyridinyl and napthyl.

The term aryloxy in accordance with the specification is not particularly limited and should be known to a person of skill in the art. The term “aryloxy” refers to aryl group, as described herein, attached to an oxygen atom. Example of an aryloxy can include, without limitation, phenoxy.

The term alkylamino in accordance with the specification is not particularly limited and should be known to a person of skill in the art. The term “alkylamino” refers to an alkyl group, as described herein, attached to an amino group. Example of an alkylamino can include, without limitation, methylamino, ethylamino and the like.

The term arylamino in accordance with the specification is not particularly limited and should be known to a person of skill in the art. The term “arylamino” refers to an aryl group, as described herein, attached to an amino group. Example of an arylamino can include, without limitation, phenylamino and the like.

The term silyloxy in accordance with the specification is not particularly limited and should be known to a person of skill in the art. The term “silyloxy” refers to a silicon atom bonded to an oxygen atom. The silicon atom can have other substituents attached to it. Example of an silyloxy can include, without limitation, trimethylsilyloxy (TMS-O), tert-butyldiphenylsilyloxy (TBDPS-O), tert-butyldimethylsilyloxy (TMDMS-O), triisopropylsilyloxy (TIPS-O) and the like.

The term hydrogenation refers to addition of hydrogen in the presence of a catalyst. In an embodiment in accordance with the specification, the hydrogenation is performed to reduce the benzyl carbon atom, for example and without limitation, the benzyl carbon atom of a compound of formula II. Example of catalytic hydrogenation is disclosed by, for example, Plattner, P. A. et al. in Helvetica Chemica Acta, 1949, p. 2464-74. The hydrogenation in accordance with the specification can be carried out in the presence of a solvent.

The term catalyst in accordance with the specification is not particularly limited and should be known to a person of skill in the art or can be determined. In one embodiment, the catalyst used for hydrogenation in accordance with the process of the specification is Pd/C. In another embodiment, Pd/C (10 mole %) can be used for the hydrogenation step.

The solvent for use in the hydrogenation step in accordance with the specification is not particularly limited and should be known to a skilled person or can be determined. The solvent used in accordance with the description is compatible with hydrogenation conditions, and non-reactive with hydrogen in the presence of a catalyst. In one embodiment, the solvent for hydrogenation is, for example and without limitation, methanol, ethanol, dioxane, tetrahydrofuran, isopropanol, toluene or ethyl acetate.

The hydroxyl protecting group in accordance with the specification is not particularly limited and should be known to a person of skill in the art or can be determined. In one embodiment the hydroxyl protecting group is, for example and without limitation, benzyl, p-methoxybenzyl ether (PMB), diphenylmethyl ether, 1-pyrenylmethyl ether and the like. In another embodiment, the hydroxyl protecting group is —CH₂Ar.

In an embodiment in accordance with the specification, a base can be used to carry out the reaction. The base used is not particularly limited and should be known to a person of skill in the art or can be determined. In one embodiment, the base is, for example and without limitation, sodium hydride, triethylamine, pyridine, imidazole and the like.

The term chiral resolution used in accordance with the specification is not particularly limited, and should be known to a person of skill in the art or can be determined. In one embodiment, for example and without limitation, chiral resolution is carried out by chiral salt formation, chiral chromatographic separation or chiral enzymatic hydrolysis. In another embodiment, for example and without limitation, chiral resolution is performed by formation of a chiral diastereomeric salt, for example and without limitation, salt formation with tartaric acid, followed by separation, for example and without limitation, by precipitation or recrystallization. In another embodiment, the chiral resolution can be performed using an enzyme to perform stereoselective hydrolysis. The enzyme can be for example, and without limitation, a lipase or any esterase known to the person skilled in the art and that perform stereoselective hydrolysis.

In another aspect, the specification relates to a compound of formula II, as shown below. The substituents R¹, R², R³, X, Y, Z and [symbol] are as described above.

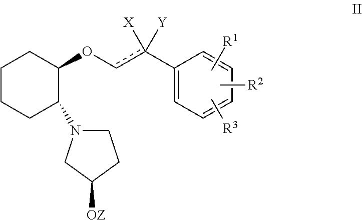

In another aspect, the specification relates to a compound of formula II′, as shown below. The substituents X, Y, Z and [symbol] are as described above. The compound of formula II′ can be prepared from readily available starting materials. In addition, the compound of formula II or II′ can allow for a single global deprotection step at or near the end of the process for the preparation of a compound of formula I.

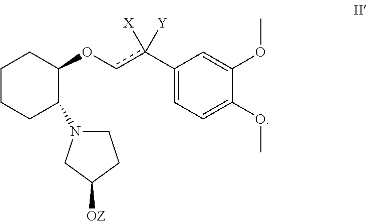

In one embodiment, the specification relates to a compound of formula IIa, as shown below, where Z is a benzyl group, and X and Y together with the carbon atom to which they are attached form a carbonyl group (C═O).

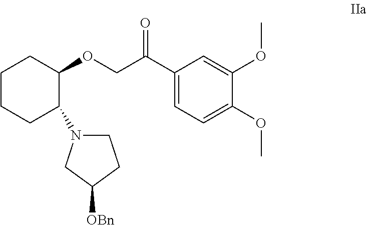 [SMILES: COc1ccc(C(=O)CO[C@@H]2CCCC[C@H]2N2CC[C@@H](OCc3ccccc3)C2)cc1OC]

In another embodiment, the specification relates to a compound of formula IIb, as shown below, where Z is a benzyl group, and one of X and Y is a hydroxyl group and the other is hydrogen.

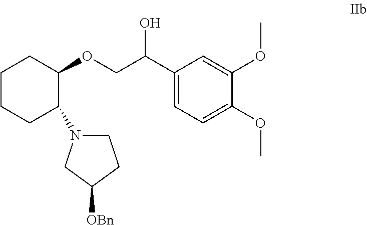 [SMILES: COc1ccc(C(O)CO[C@@H]2CCCC[C@H]2N2CC[C@@H](OCc3ccccc3)C2)cc1OC]

In another embodiment, the specification relates to a compound of formula IIc, as shown below, where Z is a benzyl group, and one of X and Y is —OTBS and the other is hydrogen.

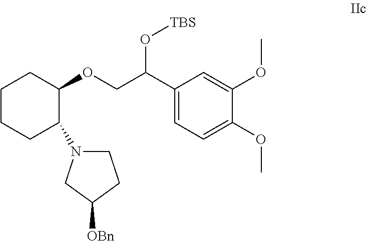

In another aspect, the specification relates to a process for preparing a compound of formula II, as described above, where X and Y are H and OH, or vice versa. The process is as disclosed in Scheme 1, where a compound of formula III, as described above, is reacted with a compound of formula IV, as described above.

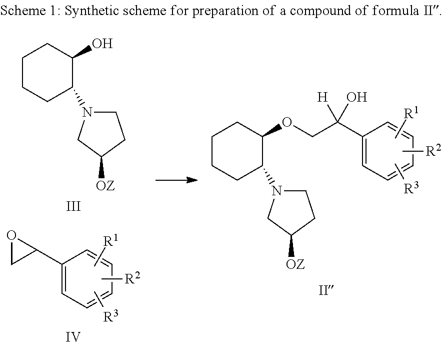

In one embodiment in accordance with the specification, the compound of formula III is prepared as shown in Scheme 2, where a cyclohexyl epoxide of formula V, as described above, is reacted with a compound of formula VI, as described above, followed by chiral resolution, allowing preparation of a compound of formula III.

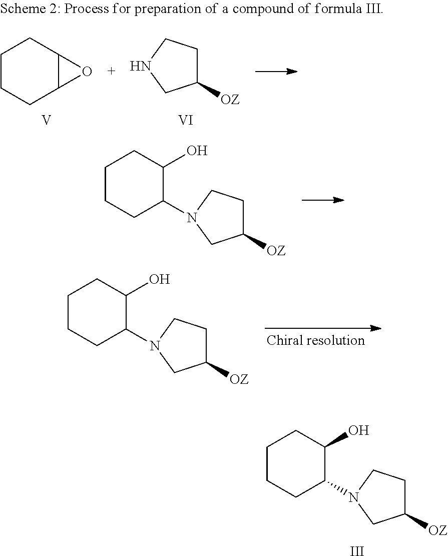

### EXAMPLES

The following examples are illustrative and non-limiting and represent specific embodiments of the present invention.

General

All NMR spectra were obtained on a Bruker Avance II, 300 MHz model. Coupling constants given for NMR are in Hz. Mass spectral data was acquired using an Agilent 6330 Ion Trap or a Bruker Daltonics MicrOTOF instrument. Temperatures reported are for that of the bath. RT indicates room temperature of approximately 15 to 30° C. DCM is short for dichloromethane.

### Example 1

### Preparation of Compound IIb

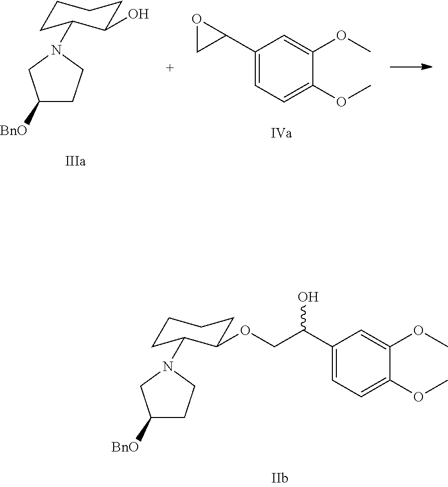 [SMILES: OC1CCCCC1N1CC[C@@H](OCc2ccccc2)C1 ; COc1ccc(C2CO2)cc1OC ; COc1ccc(C(O)COC2CCCCC2N2CC[C@@H](OCc3ccccc3)C2)cc1OC]

To a dry, nitrogen purged 200 mL round bottomed flask equipped with a magnetic stir bar was added NaH (60% dispersion in mineral oil, 1.23 g, 30.7 mmol). The flask was cooled in an ice bath. To the flask was added sequentially, anhydrous tetrahydrofuran (THF) (60 mL), hexamethylphosphoramide (HMPA) (9 mL) and IIIa (4.51 g, 16.4 mmol), then the mixture heated at 45° C. for 30 min. After cooling to RT, 3,4-dimethoxystyrene oxide (IVa) (3.08 g, 17.1 mmol) was added and the mixture heated at 55° C. for 17 h, then 70° C. for 23 h. The mixture was cooled to RT, then NaH was added (60% dispersion in mineral oil, 0.22 g, 5.5 mmol). After stirring at RT for 75 min, 3,4-dimethoxystyrene oxide (IVa) (2.38 g, 13.2 mmol) was added as a solution in anhydrous THF (3 mL). The mixture was stirred at RT for 20 min; then heated at 74° C. for 29 h. After cooling to RT, the reaction was quenched by addition of ammonium chloride NH₄Cl (sat. aq). The mixture was extracted with methyl tert-butyl ether (MTBE) (2×50 mL). The combined extracts were washed with water (2×50 mL), brine (1×50 mL), dried over sodium sulphate (Na₂SO₄), filtered and concentrated in vacuo to give a dark oil. Chromatographic purification was achieved using a Biotage system (KP—SIL; eluent A: 2% iPr₂NH in EtOAc, eluent B: heptanes; gradient elution: 20% A to 100% A over 12 column volumes) to give IIa. [MS (ES) 456.3 [M+1] (100%)] as two diastereomers, IIb-d1 and IIb-d2.

Characterization of IIb-d1: ¹H NMR (300 MHz, CDCl₃) δ 7.32 (m, 5H), 6.96 (d, J=1.5 Hz, 1H), 6.89 (dd, J=8.3, 1.5, 1H), 6.81 (d, J=8.3, 1H), 4.74 (dd, J=8.9, 3.0, 1H), 4.50 (d, J=16.1, 1H), 4.46 (d, J=16.1, 1H), 4.20 (m, 1H), 3.87 (s, 3H), 3.84 (s, 3H), 3.56 (dd, J=11.0, 3.1, 1H), 3.46 (m, 1H), 3.37 (td, J=10.0, 4.2, 1H), 3.14 (dd, J=9.8, 6.3, 1H), 2.92 (dd, J=16.4, 8.3, 1H), 2.81-2.73 (m, 1H), 2.68 (dd, J=9.8, 4.1, 1H), 2.15-1.99 (m, 2H), 1.93-1.85 (m, 2H), 1.74 (m, 2H), 1.36-1.17 (m, 4H).

Characterization of IIb-d2: ¹H NMR (300 MHz, CDCl₃) δ 7.32 (m, 5H), 6.92 (d, J=1.5, 1H), 6.85-6.80 (m, 2H), 4.80 (dd, J=9.3, 2.6, 1H), 4.52 (d, J=16.0, 1H), 4.48 (d, J=16.0, 1H), 4.23 (m, 1H), 3.94 (dd, J=10.6, 2.7, 1H), 3.85 (s, 6H), 3.27-3.20 (m, 2H), 3.12 (dd, 9.9, 6.2, 1H), 2.97-2.76 (m, 3H), 2.68-2.61 (m, 1H), 2.20-2.04 (m, 2H), 1.94-1.86 (m, 2H), 1.74 (br s, 1H), 1.37-1.18 (m, 4H).

### Example 2

### Preparation of 1R-{2R-[2-(3,4-dimethoxyphenyl)-ethoxy]-cyclohexyl}-pyrrolidin-3R-ol (Ia)

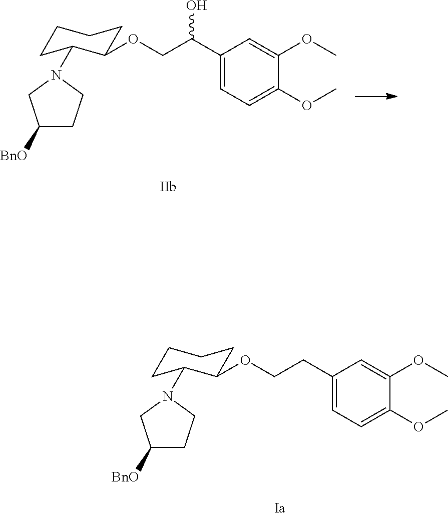 [SMILES: COc1ccc(C(O)COC2CCCCC2N2CC[C@@H](OCc3ccccc3)C2)cc1OC ; COc1ccc(CCOC2CCCCC2N2CC[C@@H](OCc3ccccc3)C2)cc1OC]

To a stirring solution of IIb (142 mg, 0.312 mmol) in methanol (MeOH) (10 mL) under nitrogen was added 2M HCl aq (1 mL), then 10% Pd/C (126 mg). The reaction flask was evacuated and purged with hydrogen three times and left under hydrogen for about 3 h. The reaction flask was evacuated and purged with nitrogen three times, then the mixture filtered through celite, rinsing with MeOH. To the filtrate was added NaHCO₃ aq (20 mL) and then concentrated to remove MeOH. The aqueous mixture was then extracted with MTBE (2×10 mL). The combined extracts were washed with brine (10 mL), dried with Na₂SO₄, filtered and concentrated in vacuo to give Ia (50 mg) as a colorless film.

Characterization of Ia: ¹H NMR (300 MHz, CDCl₃) δ 6.75 (m, 3H), 4.22 (m, 1H), 3.87 (s, 3H), 3.85 (m, 3H), 3.74 (m, 1H), 3.57 (m, 1H), 3.32 (td, J=7.7, 3.5, 1H), 2.96-2.75 (m, 5H), 2.64 (dd, J=10.0, 5.0, 1H), 2.49-2.37 (m, 2H), 2.05-1.98 (m, 2H), 1.84 (m, 1H), 1.69-1.62 (m, 3H), 1.35-1.19 (m, 4H). MS (ES) 350.2 [M+1] (100%)

### Example 3

### Preparation of Compound IIc and Conversion to Ia

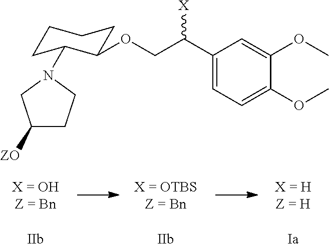

A stirring solution of IIb (0.54 g, 1.2 mmol) in anhydrous dichloromethane (DCM) (5 mL) under nitrogen was treated with imidazole (210 mg, 3.08 mmol) and tert-butyldimethylsilyl chloride (TBSCI) (515 mg, 3.42 mmol). The mixture was stirred at RT for 16 h, then diluted with water and DCM. The DCM layer was separated, then the aqueous cut extracted again with DCM. The combined extracts were dried over Na₂SO₄, filtered and concentrated in vacuo to give a yellow oil. Chromatographic purification was achieved using a Biotage system (KP—NH; eluent A: EtOAc, eluent B: heptanes; gradient elution 5% A to 20% A over about 12 column volumes) to give IIc.

Characterization of IIc: ¹H NMR (300 MHz, CDCl₃) δ 7.32 (m, 5H), 6.94 (d, J=1.4, 1H), 6.85 (dd, J=8.3, 1.4, 1H), 6.77 (d, J=8.3, 1H), 4.74 (dd, 7.1, 4.5, 1H), 4.47 (d, J=15.3, 1H), 4.43 (d, J=15.4, 1H), 4.04 (m, 1H), 3.86 (s, 3H), 3.85 (s, 3H), 3.56 (dd, J=9.4, 7.3, 1H), 3.41 (dd, J=9.4, 4.6, 1H), 3.33 (m, 1H), 2.83 (dd, J=9.8, 6.5, 1H), 2.72-2.51 (m, 3H), 2.28 (m, 1H), 2.02-1.73 (m, 4H), 1.60 (m, 4H), 1.37-1.19 (m, 2H), 0.88 (s, 9H), 0.00 (s, 6H). MS (ES) 570.4 [M+1] (100).

To a stirring solution of IIc (369 mg, 0.648 mmol) in MeOH (15 mL) under nitrogen was added 2M HCl aq (1 mL), then 10% Pd/C (206 mg). The reaction flask was evacuated and purged with hydrogen three times and left under hydrogen for about 19 h. The reaction flask was evacuated and purged with nitrogen three times, then the mixture filtered through celite, rinsing with MeOH. To the filtrate was added NaHCO₃ aq (20 mL) and then concentrated to remove MeOH. The aqueous mixture was then extracted with MTBE (2×10 mL). The combined extracts were washed with brine (10 mL), dried with Na₂SO₄, filtered and concentrated in vacuo to give Ia (105 mg) as a colorless film.

### Example 4

### Preparation of Compound IIa

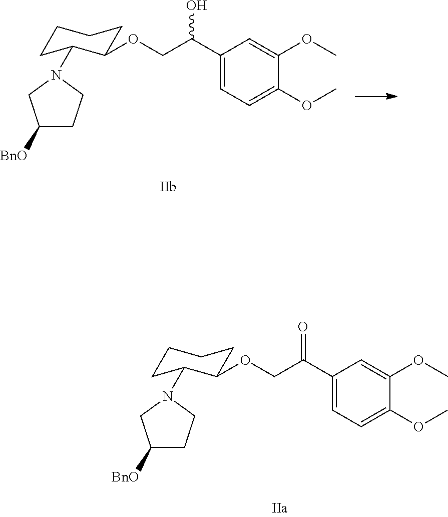 [SMILES: COc1ccc(C(O)COC2CCCCC2N2CC[C@@H](OCc3ccccc3)C2)cc1OC ; COc1ccc(C(=O)COC2CCCCC2N2CC[C@@H](OCc3ccccc3)C2)cc1OC]

To a dry, nitrogen purged round bottomed flask equipped with a magnetic stir bar was added anhydrous DCM (5 mL), then cooled in a dry ice-acetone bath. To the flask was sequentially added oxalyl chloride (0.07 mL, 0.8 mmol) and anhydrous dimethyl sulphoxide (DMSO) (0.08 mL, 1.1 mmol). The mixture was stirred for 25 min, then a solution of IIb (232 mg, 0.510 mmol) in DCM (2 mL) was added dropwise. After stirring for about 40 min, triethylamine (TEA) (0.35 mL, 2.5 mmol) was added, then the mixture allowed to warm to RT, diluted with DCM and water (−5 mL each) and the layers separated. The aqueous cut was extracted again with DCM, then the combined organics washed with water (1×5 mL), dried with Na₂SO₄, filtered and concentrated in vacuo. Chromatographic purification of the residue was achieved using a Biotage system (KP—NH; eluent A: EtOAc, eluent B: heptanes; gradient elution 0% A to 50% A over about 8 column volumes) to give IIa. (20 mg).

Characterization of IIa: ¹H NMR (300 MHz, CDCl3) δ 7.64 (dd, J=8.4, 1.8, 1H) 7.55 (d, J=1.8, 1H), 7.29 (m, 5H), 6.84 (d, J=8.4, 1H), 4.83 (d, J=16.0, 1H), 4.75 (d, J=16.0, 1H), 4.45 (s, 2H), 4.04 (m, 1H), 3.92 (s, 3H), 3.91 (s, 3H), 3.45 (m, 1H), 2.92 (dd, J=9.8, 6.3, 1H), 2.82-2.75 (m, 2H), 2.60-2.51 (m, 2H), 2.02-1.98 (m, 2H), 1.85-1.81 (m, 3H), 1.66 (m, 2H), 1.46-1.24 (m, 3H). MS (ES) 454.3 [M+1] (100).

### Example 5

### Preparation of Compound IId

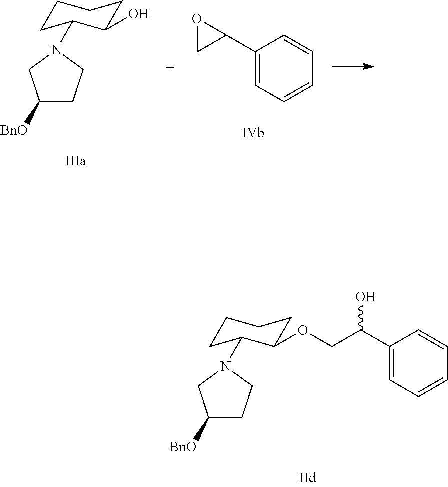 [SMILES: OC1CCCCC1N1CC[C@@H](OCc2ccccc2)C1 ; c1ccc(C2CO2)cc1 ; OC(COC1CCCCC1N1CC[C@@H](OCc2ccccc2)C1)c1ccccc1]

To a dry, nitrogen purged 250 mL round bottomed flask equipped with a magnetic stir bar was added NaH (60% dispersion in mineral oil, 0.461 g, 11.5 mmol). The flask was cooled in an ice bath. To the flask was added sequentially, anhydrous THF (40 mL), HMPA (10 mL) and IIIa (3.015 g, 10.96 mmol, rinsing with 5 mL THF), then the mixture heated at 65-70° C. for 10 min. After cooling to RT, styrene oxide (IVb) (1.30 mL, 11.4 mmol) was added, the mixture stirred at RT for 20 min, then heated at 60° C. for about 24 h. After cooling to RT, the reaction was quenched by addition to NH₄Cl (sat. aq, 50 mL). The mixture was diluted with water (10 mL); then extracted with MTBE (2×50 mL). The combined extracts were washed with water (2×50 mL), brine (1×30 mL), dried over Na₂SO₄, filtered and concentrated in vacuo to give a dark oil. Chromatographic purification was achieved using a Biotage system (KP—SIL; eluent A: 2% iPr2NH in EtOAc, eluent B: heptanes; gradient elution: 20% A to 100% A over 12 column volumes) to give IId (88 mg) as inseparable diastereomers (yellow oil).

Characterization of IId: ¹H NMR (300 MHz, CDCl3) δ 7.34 (m, 10H), 4.80 (m, 1H), 4.50 (m, 2H), 4.22 (m, 1H), 3.61 (m, 3H), 3.18 (m, 1H), 2.93-2.72 (m, 4H), 2.03-1.75 (m, 6H), 1.24 (m, 4H). MS (ES) 276.2 [M+1]-C8H80 (43), 396.3 [M+1] (44), 516.3 [M+1]+C8H80 (100).

### Example 6

### Preparation of Ic

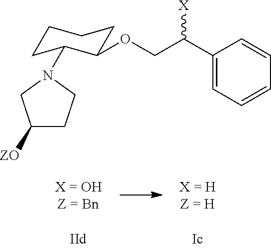

To a stirring solution of IId (202 mg, 0.51 mmol) in MeOH (10 mL) under nitrogen was added 2M HCl aq (1 mL), then 10% Pd/C (129 mg). The reaction flask was evacuated and purged with hydrogen three times and left under hydrogen for about 2 h. The reaction flask was evacuated and purged with nitrogen three times; then the mixture filtered through celite, rinsing with MeOH. To the filtrate was added NaHCO₃ aq (10 mL) and then concentrated to remove MeOH. The aqueous mixture was then extracted with MTBE (2×10 mL). The combined extracts dried over Na₂SO₄, filtered and concentrated in vacuo to give Ic (88 mg) as a yellow oil.

Characterization of Ic: ¹H NMR (300 MHz, CDCl3) δ 7.21 (m, 5H), 4.17 (m, 1H), 3.80-3.74 (m, 1H), 3.61-3.54 (m, 1H), 3.28 (td, J=8.1, 3.6, 1H), 2.91-2.83 (m, 4H), 2.71 (d, J=10, 1H), 2.59 (dd, J=10.0, 5.1, 1H), 2.47-2.35 (m, 2H), 2.10-1.96 (m, 2H), 1.85-1.59 (m, 4H), 1.32-1.15 (m, 4H). MS (ES) 290.2 [M+1] (100).

### Example 7

### Preparation of Compound IIIa

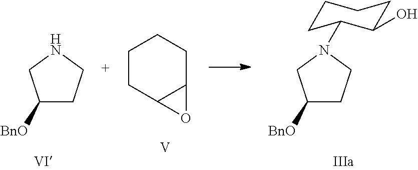

A 250 mL round bottomed flask was charged with 3R-benzyloxypyrrolidine VI′, (16.82 g, 95.9 mmol), cyclohexene oxide V (12.01 g, 122.4 mmol) and 7.1 mL of DI water. The mixture was heated at about 85° C. for about 6 hours, then cooled to RT, diluted with water (12 mL) and adjusted to pH of about 4 with 1M HCl (aq). The acidic mixture was washed with MTBE (3×37 mL); then the pH adjusted to about 9. The basic mixture was extracted with MTBE (3×50 mL) and then the extracts combined and concentrated to give a dark brown oil. The crude oil (17.4 g, 63.2 mmol) dissolved in ethyl acetate (139 mL) was treated with (−)-O,O′-ditoluoyl-L-tartaric acid (12.23 g, 31.65 mmol) and stirred at RT for about 16 h, then the solid was collected by filtration, washed with ethyl acetate (2×30 mL) and dried to give the amine-tartrate salt (26.17 g, 27.94 mmol). To a slurry of the solid in toluene (262 mL) was added DI water (131 mL) and NaOH (2.4 g, 59.38 mmol). The mixture was stirred at RT for about 1 h and then the layers separated. The aqueous cut was extracted again with toluene (1×50 mL), then the organic layers combined and concentrated to give IIIa as an oil (8.95 g).

Characterization of IIIa: ¹H NMR (300 MHz, CDCl3) 7.32-7.22 (m, 5H), 4.46 (d, J=12.0, 1H), 4.42 (d, J=12.0, 1H), 4.10-4.03 (m, 1H), 3.84 (br s, 1H), 3.35-3.27 (m, 1H), 2.95 (dd, J=9.8, 6.2, 1H), 2.86 (dd, J=15.9, 7.7, 1H), 2.65-2.52 (m, 2H), 2.43-2.36 (m, 1H), 2.11-1.97 (m, 2H), 1.86-1.68 (m, 4H), 1.26-1.13 (m, 4H). MS (ES) 276.2 [M+1] (100%).

### Example 9

### Preparation of Ia

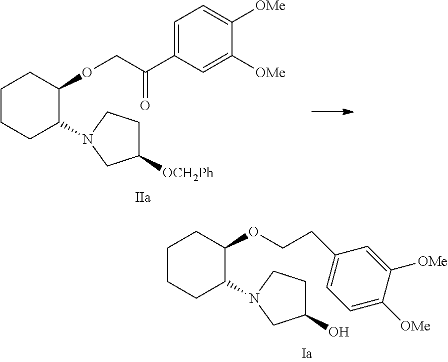

To a stirring solution of IIa (88 mg, 0.19 mmol) in MeOH (3.6 mL) under nitrogen was added 10% Pd/C (54 mg). The reaction flask was evacuated and purged with hydrogen three times and left under hydrogen for about 2 h, then charged with 2M HCl aq (0.36 mL). Additional 10% Pd/C was charged (102 mg) and the reaction stirred under hydrogen until judged complete by TLC. The reaction flask was evacuated and purged with nitrogen three times, then the mixture filtered through celite, rinsing with MeOH. To the filtrate was added NaHCO3 aq (4 mL) and then concentrated to remove MeOH. The aqueous mixture was then extracted with MTBE (2×4 mL), then DCM (2×4 mL). The MTBE extracts were combined, dried with Na2SO4, filtered and concentrated in vacuo to give Ia as a colorless oil. The DCM extracts were combined, dried with Na2SO4, filtered and concentrated in vacuo to give Ia as a colorless oil.

Following the methodology disclosed, a number of different compounds in accordance with the specification can be prepared. Table 1 provides, as an example, a list of different substituents that can be present in the compound of formula I and which can be prepared in accordance with the specification.

TABLE 1
Substituents on compound of formula I

| | R¹ | R² | R³ | R⁴ | R⁵ | R⁶ | R⁷ |
|---|---|---|---|---|---|---|---|
| A | CH₃ | H | H | — | — | — | — |
| B | CH₃ | OCH₃ | H | — | — | — | — |
| C | H | H | N(R⁴)R⁵ | H | C(O)CH₃ | — | — |
| D | CH₃ | SO₂N(R⁶)R⁷ | H | — | — | H | H |

1. A process for preparation of aminocyclohexyl ether of formula I 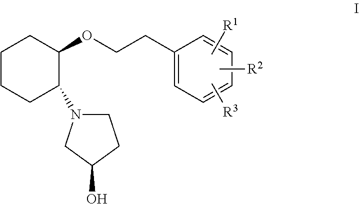
or a pharmaceutically acceptable salt, ester, or prodrug thereof; wherein:
R¹, R² and R³ each independently is bromine, chlorine, fluorine, carboxy, hydrogen, hydroxy, hydroxy methyl, methanesulfonamido, cyano, sulfamyl, trifluoromethyl, —CHF₂, —SO₂N(R⁶)R⁷, —OCF₃, C₁₋₆alkyl, C₁₋₆alkoxy, C₂₋₇alkoxycarbonyl, C₂-C₇alkanoyloxy, aryl or —N(R⁴)R⁵;
with the proviso that at least one of R¹, R² or R³ is other than hydrogen; and
R⁴, R⁵, R⁶ or R⁷ each independently is hydrogen, acetyl, methanesulfonyl or C₁₋₆alkyl;
the process comprising:
hydrogenating, in the presence of a catalyst, a compound of formula II 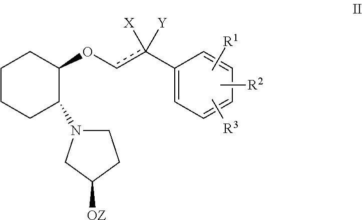
wherein X and Y each independently is hydrogen, hydroxyl, amino, C₁₋₆alkoxy, C₆₋₁₄aryloxy, C₁₋₆alkylamino, C₆₋₁₄arylamino, or silyloxy, or X and Y together with the carbon atom to which they are attached form C═O, with the proviso that at least one of X and Y is other than hydrogen;
Z is a hydroxyl protecting group; and [symbol]
is a single or double bond, and wherein when [symbol] is a double bond, one of X or Y is absent and the one of X or Y present is other than hydrogen; and
optionally converting the aminocyclohexyl ether of formula I into its pharmaceutically acceptable salt, ester, or prodrug thereof.

2. The process according to claim 1, wherein the catalyst is Pd/C.

3. The process according to claim 1, wherein the catalyst is Pd/C (10 mole %).

4. The process according to claim 1, wherein Z is —CH₂Ar, wherein Ar is an aryl group.

5. The process according to claim 1, wherein Z is —CH₂Ph.

6. The process according to claim 1, further comprising
reacting a compound of formula III 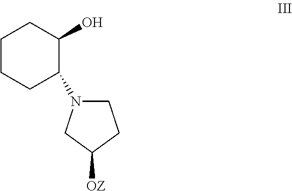
with an epoxide of formula IV 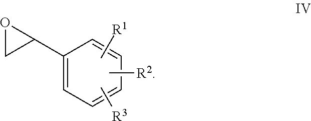
to form the compound of formula II, and
optionally silylating the reaction product to form the silyloxy group.

7. The process according to claim 6, wherein the reaction is carried out in the presence of a base.

8. The process according to claim 7, wherein the base is sodium hydride.

9. The process according to claim 6, wherein the compound of formula III is prepared by reacting a cyclohexyl epoxide of formula V 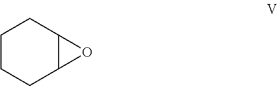
with a compound of formula VI 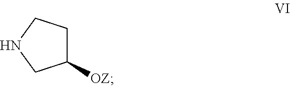
and performing chiral resolution of the diastereomeric mixture to form the compound of formula III.

10. The process according to claim 1, wherein the compound of formula I prepared is a compound of formula Ia

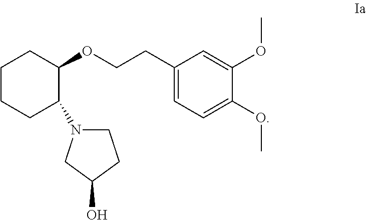 [SMILES: COc1ccc(CCO[C@@H]2CCCC[C@H]2N2CC[C@@H](O)C2)cc1OC]

11. A compound of formula II 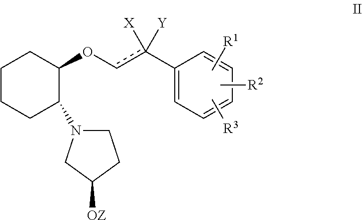
wherein R¹, R² and R³ each independently is bromine, chlorine, fluorine, carboxy, hydrogen, hydroxy, hydroxy methyl, methanesulfonamido, cyano, sulfamyl, trifluoromethyl, —CHF₂, —SO₂N(R⁶)R⁷, —OCF₃, C₁₋₆alkyl, C₁₋₆ alkoxy, C₂₋₇alkoxycarbonyl, C₂-C₇alkanoyloxy, aryl or —N(R⁴)R⁵; with the proviso that at least one of R¹, R² or R³ is other than hydrogen; and
R⁴, R⁵, R⁶ or R⁷ each independently is hydrogen, acetyl, methanesulfonyl or C₁₋₆alkyl;
X and Y each independently is hydrogen, hydroxyl, amino, C₁₋₆alkoxy, C₆₋₁₄aryloxy, C₁₋₆alkylamino, C₆₋₁₄arylamino, or silyloxy, or X and Y together with the carbon atom to which they are attached form C═O, with the proviso that at least one of X and Y is other than hydrogen;
Z is a hydroxyl protecting group; and [symbol]
is a single or double bond, and wherein when [symbol] is a double bond, one of X or Y is absent and the one of X or Y present is other than hydrogen.

12. (canceled)

13. (canceled)

14. (canceled)

15. (canceled)

16. A process for preparing a compound of formula II, 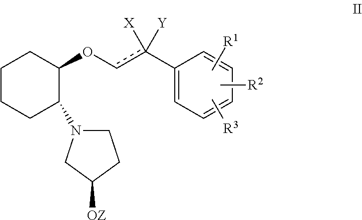
wherein R¹, R² and R³ each independently is bromine, chlorine, fluorine, carboxy, hydrogen, hydroxy, hydroxy methyl, methanesulfonamido, cyano, sulfamyl, trifluoromethyl, —CHF₂, —SO₂N(R⁶)R⁷, —OCF₃, C₂-C₇alkanoyloxy,
C₁₋₆alkyl, C₁₋₆alkoxy, C₂₋₇alkoxycarbonyl, aryl or —N(R⁴)R⁵; with the proviso that at least one of R¹, R² or R³ is other than hydrogen; and
R⁴, R⁵, R⁶ or R⁷ each independently is hydrogen, acetyl, methanesulfonyl or C₁₋₆alkyl;
X and Y each independently is hydrogen, hydroxyl, amino, C₁₋₆alkoxy,
C₆₋₁₄aryloxy, C₁₋₆alkylamino, C₆₋₁₄arylamino, or silyloxy, or X and Y together with the carbon atom to which they are attached form C═O, with the proviso that at least one of X and Y is other than hydrogen;
Z is a hydroxyl protecting group; and [symbol]
is a single or double bond, and wherein when [symbol] is a double bond, one of X or Y is absent and the one of X or Y present is other than hydrogen;
the process comprising:
reacting a compound of formula III 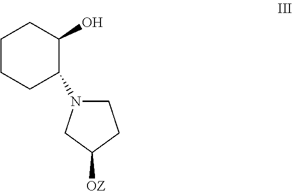
wherein Z is a protecting group, with an epoxide of formula IV 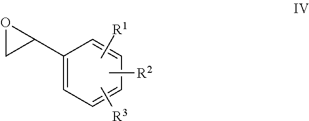
optionally silylating the reaction product to form the silyloxy group.

17. The process according to claim 16, wherein the compound formula III is prepared by reacting a cyclohexyl epoxide of formula V 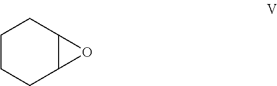
with a compound of formula VI, followed by chiral resolution

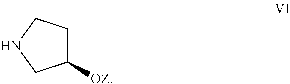

18. The process according to claim 16, wherein R¹ is H, R² is —OCH₃ and R³ is —OCH₃, and the compound of formula II prepared is a compound of formula II′ [chemical structure]
